(19) United States
(12) Patent Application Publication
(10) Pub. No.: US 2022/0170925 A1
(43) Pub. Date: Jun. 2, 2022

(54) SURFACE PLASMON RESONANCE SENSOR CHIP HAVING SENSOR SURFACE CAPABLE OF CAPTURING MULTIPLE SPECIES OF ANTIBODIES AND METHOD OF MAKING

(71) Applicant: BIO-TECHNE CORPORATION, Minneapolis, MN (US)

(72) Inventor: Robyn Ann Stoller, Minneapolis, MN (US)

(21) Appl. No.: 17/674,623

(22) Filed: Feb. 17, 2022

Related U.S. Application Data
(62) Division of application No. 16/319,056, filed on Jan. 18, 2019.
(60) Provisional application No. 62/363,997, filed on Jul. 19, 2016.

(51) Int. Cl. G01N 33/543; G01N 33/553; G01N 33/68

(52) U.S. Cl. CPC G01N 33/54373; G01N 33/6854; G01N 33/553

(57) ABSTRACT

A surface plasmon resonance chip includes a sensor surface having a substrate, an inert metal layer directly on the substrate, a surface layer directly on the inert metal layer, and fusion proteins immobilized on the surface layer. The fusion proteins can be selected from A/G fusion proteins, A/L, fusion proteins, G/L fusion proteins and A/G/L fusion proteins.

### CROSS-REFERENCE

This application claims priority to U.S. Provisional Application No. 62/363,997, filed Jul. 19, 2016, the entire contents of which are incorporated herein by reference.

### BACKGROUND

Existing surface plasmon resonance sensor chips include sensor surfaces. If it is desired to immobilize a particular species of antibody on a sensor surface, the sensor surface must be treated with a protein that is specific for that antibody. This is a cumbersome and time consuming process. It would be desirable to provide a surface plasmon resonance sensor chip that includes a sensor surface that can capture more than one species of antibody. It would be particularly desirable to provide a surface plasmon resonance sensor chip that includes a sensor surface that can capture any species of antibody.

### BRIEF DESCRIPTION OF DRAWINGS

FIG. 1 illustrates a sensor surface according to one embodiment.

FIG. 2 illustrates a sensor chip with a sensor surface according to one embodiment.

FIG. 3 illustrates a method of making a sensor surface according to one embodiment.

FIG. 4 illustrates a method of making a sensor surface including A/G/L fusion proteins according to one embodiment.

FIG. 5 illustrates a process of capturing an antibody and antigen using a sensor surface including A/G/L fusion proteins according to one embodiment.

FIG. 6 illustrates charts showing responses of each a mouse, rabbit, rat and human antibody using a sensor surface including A/G/L fusion proteins.

FIG. 7 illustrates a chart showing stability of a sensor surface including A/G/L fusion proteins.

FIG. 8 illustrates a chart showing antibody species that can be used in embodiments where the sensor surface includes A/G/L fusion proteins.

### DETAILED DESCRIPTION

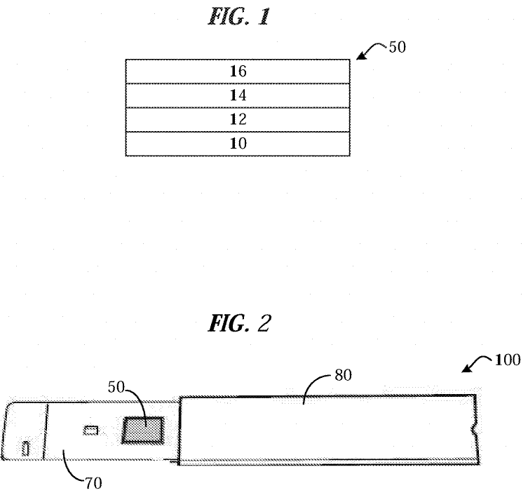

Certain embodiments provide a sensor surface 50 for a surface plasmon resonance chip. FIG. 1 illustrates a sensor surface 50 according one embodiment. The sensor surface 50 comprises a substrate 10 and a series of layers. In some cases, the sensor surface 50 comprises, moving outwardly from the substrate 10, a metal layer 12, a surface layer 14 and a protein layer 16.

The substrate 10 can be a glass substrate in some embodiments. The metal layer 12 can be a layer of an inert metal such as gold or silver. In many embodiments, the metal layer 12 is a layer comprising, consisting essentially of or consisting of gold. Also, the metal layer 12 can be provided at a thickness in the range of 40 nm to 60 nm, such as in the range of 45 nm to 55 nm. In some cases, the metal layer 12 is provided at a thickness of approximately 50 nm. The metal layer 12 can be deposited on the substrate 10 using any suitable deposition method.

The surface layer 14 can be any layer that protects biological samples from direct contact with the metal layer 12 while also providing a matrix that allows for biological interactions to take place. In some embodiments, the surface layer 14 is a layer comprising, consisting essentially of or consisting of a flexible unbranched carbohydrate polymer. In certain embodiments, the surface layer 14 is a layer comprising, consisting essentially of or consisting of carboxymethylated dextran. Also, the surface layer 14 can be provided at a thickness in the range of 90 nm to 110 nm, such as in the range of 95 nm to 105 nm. In some cases, the surface layer 14 can be provided at a thickness of approximately 100 nm.

The protein layer 16 comprises fusion proteins. In some embodiments, the protein layer 16 comprises, consists essentially of or consists of A/G/L fusion proteins. In other embodiments, the protein layer 16 comprises, consists essentially of or consists of A/G fusion proteins. In other embodiments, the protein layer 16 comprises, consists essentially of or consists of A/L, fusion proteins. In yet other embodiments, the protein layer 16 comprises, consists essentially of or consists of G/L fusion proteins. In embodiments where the protein layer 16 includes A/G fusion proteins, A/L fusion proteins or G/L fusion proteins, more antibody species can be captured than if the protein layer included A proteins, G proteins or L proteins alone.

The embodiment where the protein layer 16 includes A/G/L fusion proteins is particularly desirable because it allows for any antibody species to be captured. FIG. 8 illustrates all of the antibody species that can be captured using the A/G/L fusion proteins embodiment. Protein A/G/L is a genetically engineered protein that combines the IgG binding profiles of all Protein A, Protein G and Protein L. It is a gene fusion product. Recombinant fusion protein A/G/L contains five Ig-binding regions of protein L, five IgG binding domains from Protein A, and two Ig-binding region of protein G. Cell wall binding regions, albumin binding regions and other non-specific binding regions have all been eliminated from the fusion protein to ensure the maximum specific IgG binding. The A/G/L fusion proteins bind to human, mouse, rat cow, goat, sheep, rabbit, guinea pig, pig, dog and cat IgG. The A/G/L fusion proteins can be A/G/L Protein 1 mg (Novus catalog number NBP2-34985) obtained from Novus Biologicals. However, A/G/L Protein is available from other sources, such as from BioVision and Amsbio. Thus, a sensor chip having the sensor surface 50 including A/G/L fusion proteins can be used in any surface plasmon resonance system wherein it is desired to capture any species of antibody. The captured antibody can then be characterized using surface plasmon resonance experiences that measure antigen binding properties, include kₐ (on-rate), k_{d} (off-rate), and K_{D} (affinity) properties.

The sensor surface 50 can be provided as part of a surface plasmon resonance sensor chip that is used in machines and systems that perform surface plasmon resonance experiments. FIG. 2 illustrates a sensor chip 100 with a sensor surface 50 according to one embodiment. The sensor chip 100 includes a sensor surface 50 that is mounted on a support 70. The sensor chip 100 can also include a protective sheath 80 that protects the sensor surface 50 when not in use. In some cases, both the support 70 and the protection sheath comprise a plastic material.

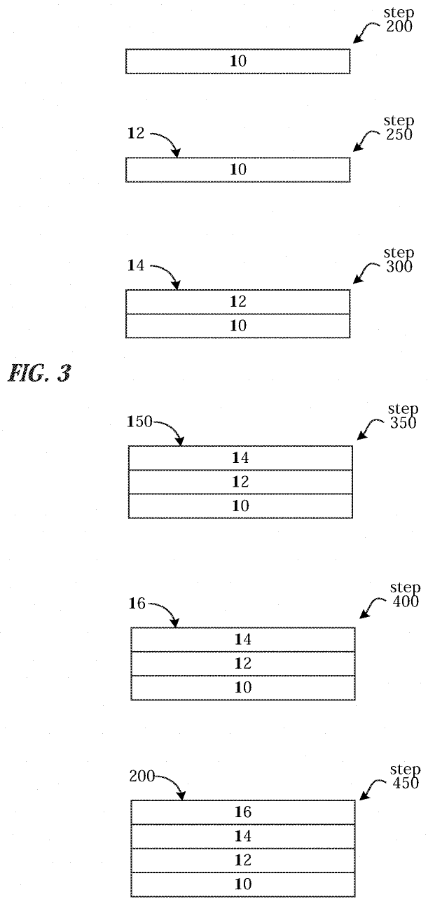

FIG. 3 illustrates a method of making a sensor surface 50 according to one embodiment. The method generally includes steps 200 through 450. A first step 200 includes providing a substrate 10, for example a glass substrate. A next step 250 includes depositing a metal layer 12 onto the substrate surface. A next step 300 includes depositing a surface layer 14. A next step 350 includes applying a surface treatment 150 to activate the surface layer 14. This activating step 350 makes certain groups active and available to attach to fusion proteins. A next step 400 includes adding fusion proteins to the activated surface layer 14. A sixth step 450 includes applying another surface treatment 200 to treat the surface layer 14 to block remaining active groups that are on the surface layer 14 but not attached to fusion proteins.

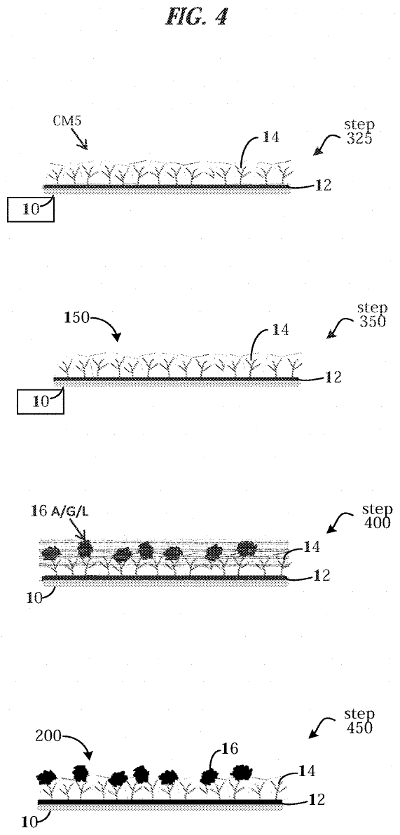

FIG. 4 illustrates a method of making a sensor surface 50 according to one particular embodiment. The method generally includes steps 325 through 450. A first step 325 includes providing a sensor surface 50 having a gold layer 12 deposited directly on a glass substrate 10 and a carboxymethyl dextran layer 14 directly on the gold layer 12. In some cases, the sensor surface 50 is part of a commercially surface plasmon resonance chip, for example a CM5 chip available from General Electric and used in instruments such as the Biacore T200 instrument. A next step 350 includes applying a surface treatment 150 to activate the carboxymethyl dextran layer 14. In certain cases, the activating step includes applying 1-ethyl-3-(3-dimethylaminopropyl)-carbodiimide (“EDC”) and N-hy droxysuccinimide (“NHS”) to make lysine groups available on the carboxymethyl dextran layer 14 to attach to the fusion proteins. A next step 400 includes adding fusion proteins 16 to the activated carboxymethyl dextran layer 14. In some cases, the fusion proteins 16 are added at a pH of less than 4, such as a pH of 3. A next step 450 applying a second surface treatment 200 to treat the carboxymethyl dextran layer 14 to block remaining lysine groups that are on the carboxymethyl dextran layer 14 but not attached to fusion proteins. In some cases, a solution comprising ethanolamine is applied to the carboxymethyl dextran layer 14 to block the remaining active groups.

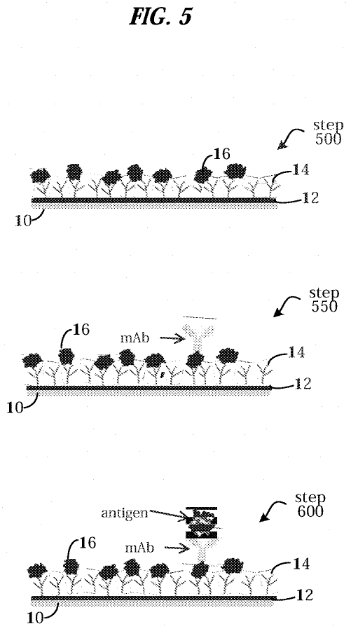

FIG. 5 illustrates a method of capturing any species of antibodies on a sensor surface 50 of a surface plasmon resonance chip according to one particular embodiment. The method includes a step 500 of providing a surface plasmon resonance chip having a sensor surface 50. The sensor surface 50 includes a substrate 10, a metal layer 12 directly on the substrate 10, a surface layer 14 directly on the metal layer, and fusion proteins A/G/L immobilized on the surface layer 14. In certain cases, the sensor surface 50 includes a glass substrate 10, a gold layer 12 directly on the glass substrate 10, a carboxymethyl dextran layer 14 directly on the gold layer 12, and fusion proteins A/G/L 16 immobilized on the carboxymethyl dextran layer 14. A next step 550 includes using the sensor surface 50 to capture any species of antibody. Step 550 can be performed using any capture mechanisms known in the art. A next step 600 includes binding an antigen to the captured antibody, also using any binding mechanisms known in the art. During this time, the sensor surface 50 is positioned within a surface plasmon resonance instrument to measure and analyze various antibody and antigen properties, including kₐ (on-rate), k_{d} (off-rate), and K_{D} (affinity) properties.

### EXAMPLE 1

One exemplary embodiment of making a sensor surface 50 will now be described. In this embodiment, a CM5 sensor chip from Biacore was obtained. The CM5 sensor chip includes a sensor surface comprising a gold layer 12 provided directly on a glass substrate 10 and a carboxymethyl dextran layer 14 provided directly on the gold layer 12. The inventors first docked a CM5 sensor chip to the Biacore T200 instrument. The inventors next performed a series of injections using the Biocore T200 instrument to treat the sensor surface 0 of the CM5 sensor chip. The series included, in order: (1) injecting 11.5 mg/ml of NHS and 75 mg/ml EDC mixture prepared immediately before use for 7 minutes; (2) injecting 100 μg/mL A/G/L fusion proteins diluted in 10 mM sodium citrate at a pH of 3.0 for 7.5 minutes; and (3) injecting 1 M ethanolamine for 7 minutes. All of the injections were injected at room temperature. Each of these steps were performed to successfully attach A/G/L fusion proteins to the sensor surface and thereafter blocking remaining active groups from the sensor surface. The A/G/L fusion proteins were an A/G/L Protein 1 mg (Novus catalog number NBP2-34985) obtained from Novus Biologicals. Such a sensor surface is capable of being used to capture any desired antibody species for use in surface plasmon resonance experiments.

### EXAMPLE 2

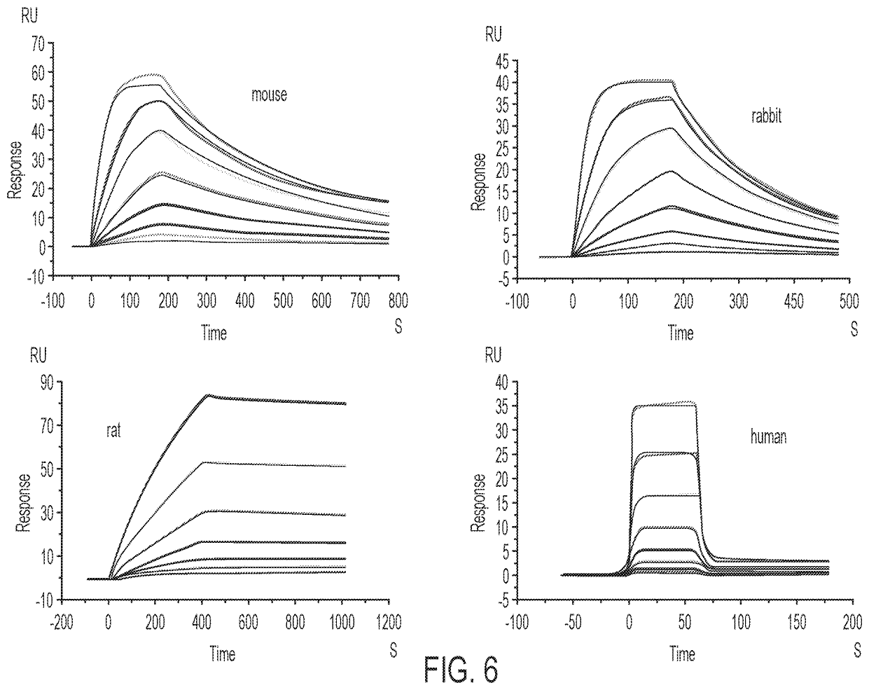

FIG. 6 illustrates charts showing responses of each a mouse, rabbit, rat and human antibody using the sensor surface 50 made according to Example 1. RU is measured over time by passing an LED light source through a glass prism, exciting the layer of gold on the surface thereby creating an energy field which reflects light based on mass changes on the surface. The angle of reflectance is converted by software in to units of response units (RU), and these data are plotted against time to provide a sensorgram. Particular graphs shown describe different concentrations of antigen injected over a surface to which antibody has been captured. Each assay is performed by capturing a fixed amount of antibody by injecting it to A/G/L sensor surface, then injecting some defined concentration range of antigen and analyzing the response. Data are fit to a 1:1 Langmuir binding model (shown). For each sensorgram, colored lines represent actual data collected while black lines represent the fit assigned by fitting the data.

### EXAMPLE 3

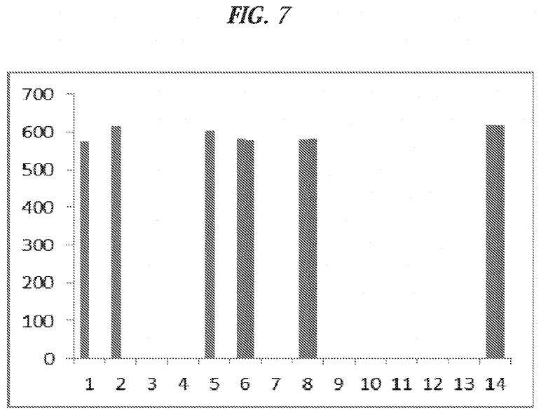

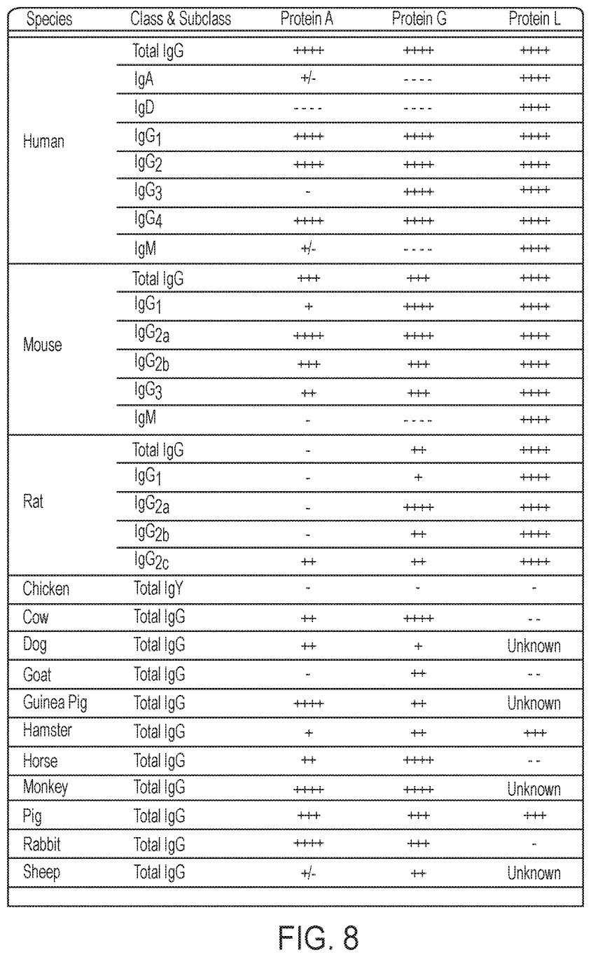

FIG. 7 illustrates a chart showing that a sensor surface 50 made according to Example 1 has excellent stability. This chart shows the total RU captured of an antibody on different days. Each day the antibody was diluted to a fixed concentration and injected for the same duration, and the response plotted over a period of days. The blue and red lines show capturing repeat injections of antibodies on the same day. Since the RU captured does not degrade or change over the 14 day test period, one would predict that this sensor surface is stable and could survive a manufacturing cycle followed by storage, allowing for potential mass production.

1. A surface plasmon resonance chip comprising a sensor surface, wherein the sensor surface comprises:
a substrate;
an inert metal layer directly on the substrate;
a surface layer directly on the inert metal layer; and
fusion proteins immobilized on the surface layer.

2. The surface plasmon resonance chip of claim 1 wherein the substrate is a glass substrate.

3. The surface plasmon resonance chip of claim 1 wherein the inert metal layer is a layer comprising gold.

4. The surface plasmon resonance chip of claim 3 wherein the inert metal layer is a layer consisting essentially of gold.

5. The surface plasmon resonance chip of claim 1 wherein the surface layer is a carboxymethyl dextran layer.

6. The surface plasmon resonance chip of claim 1 wherein the fusion proteins are selected from the group consisting of A/G fusion proteins, A/L fusion proteins, G/L fusion proteins and A/G/L fusion proteins.

7. (canceled)

8. A method of making a sensor surface comprising:
providing a substrate;
depositing a metal layer onto a surface of the substrate;
depositing a surface layer onto a surface of the metal layer; and
immobilizing fusion proteins on the surface layer, wherein the fusion proteins are fusion proteins selected from the group consisting of A/G fusion proteins, A/L, fusion proteins, G/L fusion proteins and A/G/L fusion proteins.

9. The method of claim 8 wherein the providing a substrate comprises providing a glass substrate.

10. The method of claim 8 wherein the depositing a metal layer onto a surface of the substrate comprises depositing a layer comprising gold onto a surface of the substrate.

11. The method of claim 10 wherein the layer comprising gold is a layer consisting essentially of gold.

12. The method of claim 8 wherein the depositing a surface layer onto a surface of the metal layer comprises depositing a carboxymethyl dextran layer onto a surface of the metal layer.

13. The method of claim 8 wherein the immobilizing fusion proteins on the surface layer comprises immobilizing A/G/L fusion proteins on the surface layer.

14. The method of claim 8 wherein the immobilizing fusion proteins on the surface layer comprises:
applying a first surface treatment to the surface layer to creating active groups that are capable of attaching to the fusion proteins;
adding the fusion proteins to the surface layer and allowing the fusion proteins to attach to the active groups; and
applying a second surface treatment to the surface layer to deactivate the surface layer by blocking active groups that are not attached to the fusion proteins.

15. The method of claim 14 wherein the surface layer is a carboxymethyl dextran layer and the applying the first surface treatment comprises applying 1-ethyl-3-(3-dimethylaminopropyl)-carbodiimide (“EDC”) and N-hy droxysuccinimide(“NHS”) to the carboxymethyl dextran layer to create active groups that are capable of attaching to the fusion proteins.

16. The method of claim 15 wherein the adding the fusion proteins to the surface layer and allowing the fusion proteins to attach to the active groups comprises adding the fusion proteins at a pH of less than 4, such as a pH of 3.

17. The method of claim 16 wherein the applying a second surface treatment comprises applying ethanolamine to the carboxymethyl dextran layer to deactivate the surface layer by blocking active groups that are not attached to the fusion proteins.

18. The method of claim 8 wherein the fusion proteins are A/G/L fusion proteins.

19. A method of using a surface plasmon resonance sensor chip to capture any species of antibody, comprising:
providing a surface plasmon resonance chip comprising a sensor surface, wherein the sensor surface comprises: a substrate, an inert metal layer directly on the substrate, a surface layer directly on the inert metal layer, and A/G/L fusion proteins immobilized on the surface layer; and
contacting any species of antibody with the A/G/L fusion proteins to allow the A/G/L fusion proteins to capture the antibody.

20. The method of claim 19 wherein the antibody is selected from the group consisting of human, mouse, rat cow, goat, sheep, rabbit, guinea pig, pig, dog and cat IgG.

21. The method of claim 19 wherein the sensor surface comprises a glass substrate, a gold metal layer directly on the glass substrate, a carboxymethyl dextran layer directly on the gold metal layer, and A/G/L fusion proteins immobilized on the carboxymethyl dextran layer.
